(19) United States
(12) Patent Application Publication
(10) Pub. No.: US 2026/0151572 A1
(43) Pub. Date: Jun. 4, 2026

(54) DUAL NEEDLE APPARATUS FOR MEDICATION DELIVERY

(71) Applicant: Willow Laboratories, Inc., Irvine, CA (US)

(72) Inventors: Hung The Vo, Fountain Valley, CA (US); Sai Kong Frank Lee, Irvine, CA (US); Tuan Minh Tran, Garden Grove, CA (US); Pouya Jalalmanesh, San Pedro, CA (US); Amir K. Dabiri-Asgari, Mission Viejo, CA (US); Hossein Derakhshandeh, Irvine, CA (US); Andy Peng, Irvine, CA (US); Dmitry Protsenko, Irvine, CA (US)

(21) Appl. No.: 19/354,338

(22) Filed: Oct. 9, 2025

Related U.S. Application Data
(60) Provisional application No. 63/706,265, filed on Oct. 11, 2024.

(51) Int. Cl. A61M 5/32; A61M 5/31

(52) U.S. Cl. CPC A61M 5/3298; A61M 2005/3123

(57) ABSTRACT

Systems and methods are provided for a dual needle apparatus configured to couple to a reservoir of a syringe assembly with a luer lock fitting. The dual needle apparatus may include at least a first needle and a second needle, the first needle longer than the second needle. The first needle may be configured to deliver insulin from the reservoir to the interior of a medication bladder when inserted into the medication bladder. The second needle may be configured to release air or insulin from the medication bladder to an external environment responsive the pressure caused by insulin delivery through the first needle.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### FIELD OF THE DISCLOSURE

The general field of this disclosure generally relates to glucose sensing and disease management systems.

### BACKGROUND

Diabetes is a chronic disease that impacts many individuals, both adults and children. The management of diabetes may include the measurement of glucose within the interstitial space including blood and/or interstitial fluid of a patient and administration of insulin to the patient. A closed loop insulin administration system includes both a sensor to take glucose measurements from the interstitial space including blood and/or interstitial fluid of the patient and an insulin administration device which administers insulin to the patient based on the glucose measurements. Closed loop insulin administration systems allow individuals impacted by diabetes to go about daily life with much less worry about their insulin or glucose levels which can vastly improve a diabetic's quality of life.

### SUMMARY

Various embodiments of systems, methods, and devices within the scope of the appended claims each have several aspects, no single one of which is solely responsible for the desirable attributes described herein. Without limiting the scope of the appended claims, some prominent features are described herein.

In some aspects, the techniques described herein relate to a dual needle apparatus including: a luer component, configured to couple to a reservoir to form a luer lock fitting, wherein the reservoir includes a quantity of insulin; and at least two needles of differential length, wherein the at least two needles are coupled to the luer component, wherein the at least two needles include a first needle longer than a second needle with respect to a longitudinal axis of the dual needle apparatus; wherein, when the luer component is coupled to the reservoir, the first needle is able to access the quantity of insulin, wherein the first needle is configured to deliver at least a portion of the quantity of insulin to a medication bladder; wherein an end of the second needle is positioned proximate to a vent to an external environment; and wherein, when the first needle is delivering medication to the medication bladder, the second needle vents at least one of air or insulin from the medication bladder to the external environment.

In some aspects, the techniques described herein relate to a dual needle apparatus, wherein the external environment includes air of a room environment.

In some aspects, the techniques described herein relate to a dual needle apparatus, wherein the first needle and second needle are separated by a first horizontal distance perpendicular to the longitudinal axis of the dual needle apparatus.

In some aspects, the techniques described herein relate to a dual needle apparatus, wherein a first normal vector extending from a bevel of the first needle extends in a different direction than a second normal vector extending from a bevel of the second needle.

In some aspects, the techniques described herein relate to a method including: inserting a syringe assembly into an input port of a medication bladder; the syringe assembly including: a dual needle apparatus, the dual needle apparatus including a first needle and a second needle, the first needle longer than the second needle; and a reservoir including at least a quantity of insulin; and causing at least the quantity of insulin to flow from the reservoir to the medication bladder through the first needle, wherein the second needle facilitates a release of a pressure caused by the flow of insulin through the first needle into the medication bladder at least by facilitating the release of at least one of air or insulin to an external environment.

In some aspects, the techniques described herein relate to a method, wherein inserting the syringe assembly into the input port of the medication bladder includes inserting the dual needle apparatus through a inlet seal coupled to the input port, wherein after insertion, the first needle is in communication with an interior cavity of the medication bladder and the reservoir and the second needle is in communication the interior cavity of the medication bladder and with the external environment.

In some aspects, the techniques described herein relate to a method, wherein insulin is released to an external environment through the second needle.

In some aspects, the techniques described herein relate to a method, wherein the released insulin contacts an exposed area of skin of a patient.

In some aspects, the techniques described herein relate to a method, wherein the quantity of insulin is greater than a storage capacity of the medication bladder.

In some aspects, the techniques described herein relate to a method, wherein the external environment includes air of a room environment.

In some aspects, the techniques described herein relate to a method including: depleting air from a syringe assembly, the syringe assembly including: a dual needle apparatus, the dual needle apparatus including a first needle and a second needle, the first needle longer than the second needle, and a reservoir of a syringe assembly, the reservoir configured to hold a quantity of insulin; inserting the syringe assembly into a vial, the vial including at least the quantity of insulin; and extracting the insulin from the vial to the reservoir through the first needle.

In some aspects, the techniques described herein relate to a method, wherein, when extracting the insulin from the vial, the second needle is configured to facilitate movement of the insulin from the vial at least by allowing air from an external environment to pass into the vial during extraction of the insulin, the air creating a pressure against the insulin.

In some aspects, the techniques described herein relate to a method, wherein the external environment includes air of a room environment.

In some aspects, the techniques described herein relate to a method, wherein inserting the syringe assembly into the vial includes inserting the dual needle apparatus into the vial, wherein after insertion, the first needle is in communication with an interior cavity of the vial and the reservoir.

In some aspects, the techniques described herein relate to a dual needle apparatus including: a luer component, configured to couple to a reservoir to form a luer lock fitting, wherein the reservoir includes a quantity of insulin; and at least two needles of differential length, wherein the at least two needles are coupled to the luer component, wherein the at least two needles include a first needle longer than a second needle with respect to a longitudinal axis of the dual needle apparatus; wherein, when the luer component is coupled to the reservoir, the first needle is able to access the quantity of insulin, wherein the first needle is configured to deliver at least a portion of the quantity of insulin to a medication bladder; wherein an end of the second needle is positioned proximate to a vent to an external environment; and wherein, when the first needle is delivering medication to the medication bladder, the second needle vents at least one of air or insulin from the medication bladder to the external environment.

In some aspects, the techniques described herein relate to a dual needle apparatus, wherein the external environment includes air of a room environment.

In some aspects, the techniques described herein relate to a dual needle apparatus, wherein the first needle and second needle are separated by a first horizontal distance perpendicular to the longitudinal axis of the dual needle apparatus.

In some aspects, the techniques described herein relate to a dual needle apparatus, wherein a first normal vector extending from a bevel of the first needle extends in a different direction than a second normal vector extending from a bevel of the second needle.

In some aspects, the techniques described herein relate to a method including: inserting a syringe assembly into an input port of a medication bladder; the syringe assembly including: a dual needle apparatus, the dual needle apparatus including a first needle and a second needle, the first needle longer than the second needle; and a reservoir including at least a quantity of insulin; and causing at least the quantity of insulin to flow from the reservoir to the medication bladder through the first needle, wherein the second needle facilitates a release of a pressure caused by the flow of insulin through the first needle into the medication bladder at least by facilitating the release of at least one of air or insulin to an external environment.

In some aspects, the techniques described herein relate to a method, wherein inserting the syringe assembly into the input port of the medication bladder includes inserting the dual needle apparatus through a inlet seal coupled to the input port, wherein after insertion, the first needle is in communication with an interior cavity of the medication bladder and the reservoir and the second needle is in communication the interior cavity of the medication bladder and with the external environment.

In some aspects, the techniques described herein relate to a method, wherein insulin is released to an external environment through the second needle.

In some aspects, the techniques described herein relate to a method, wherein the released insulin contacts an exposed area of skin of a patient.

In some aspects, the techniques described herein relate to a method, wherein the quantity of insulin is greater than a storage capacity of the medication bladder.

In some aspects, the techniques described herein relate to a method, wherein the external environment includes air of a room environment.

In some aspects, the techniques described herein relate to a method including: depleting air from a syringe assembly, the syringe assembly including: a dual needle apparatus, the dual needle apparatus including a first needle and a second needle, the first needle longer than the second needle, and a reservoir of a syringe assembly, the reservoir configured to hold a quantity of insulin; inserting the syringe assembly into a vial, the vial including at least the quantity of insulin; and extracting the insulin from the vial to the reservoir through the first needle.

In some aspects, the techniques described herein relate to a method, wherein, when extracting the insulin from the vial, the second needle is configured to facilitate movement of the insulin from the vial at least by allowing air from an external environment to pass into the vial during extraction of the insulin, the air creating a pressure against the insulin.

In some aspects, the techniques described herein relate to a method, wherein the external environment includes air of a room environment.

In some aspects, the techniques described herein relate to a method, wherein inserting the syringe assembly into the vial includes inserting the dual needle apparatus into the vial, wherein after insertion, the first needle is in communication with an interior cavity of the vial and the reservoir.

In some aspects, the techniques described herein relate to a dual needle apparatus including: a luer component configured to couple to a reservoir to form a luer lock fitting, wherein the reservoir includes a quantity of medication; and at least two needles of differential length, wherein the at least two needles are coupled to the luer component, wherein the at least two needles include a first needle longer than a second needle with respect to a longitudinal axis of the dual needle apparatus, wherein, when the luer component is coupled to the reservoir, the first needle is able to access the quantity of medication, wherein the first needle is configured to deliver at least a portion of the quantity of medication to a medication bladder, wherein an end of the second needle is positioned near a vent to an external environment, and wherein, when the first needle is delivering medication to the medication bladder, the second needle vents at least one of air or medication from the medication bladder to the external environment.

In some aspects, the techniques described herein relate to a dual needle apparatus, wherein the medication includes insulin.

In some aspects, the techniques described herein relate to a dual needle apparatus, wherein the external environment includes air of a room environment.

In some aspects, the techniques described herein relate to a dual needle apparatus, wherein the first needle and the second needle are separated by a horizontal distance perpendicular to the longitudinal axis of the dual needle apparatus.

In some aspects, the techniques described herein relate to a dual needle apparatus, wherein a vector extending from a bevel of the first needle extends in a different direction than a vector extending from a bevel of the second needle.

In some aspects, the techniques described herein relate to a dual needle apparatus, wherein the luer component includes a slip-tip fitting.

In some aspects, the techniques described herein relate to a dual needle apparatus, wherein the apparatus is configured to couple to a syringe assembly having a reservoir volume between 1 mL and 10 mL.

In some aspects, the techniques described herein relate to a dual needle apparatus, wherein the second needle is positioned within 5 mm of the luer component.

In some aspects, the techniques described herein relate to a dual needle apparatus, wherein the apparatus is disposable.

In some aspects, the techniques described herein relate to a dual needle apparatus, wherein the vent includes a channel extending from the second needle to an exterior surface of the apparatus.

In some aspects, the techniques described herein relate to a dual needle apparatus, wherein the apparatus is operable to deliver at least one of insulin, glucagon, or another medication.

In some aspects, the techniques described herein relate to a dual needle apparatus including: a luer component configured to couple to a reservoir; a first needle coupled to the luer component and configured to deliver fluid from the reservoir to a medication bladder; a second needle coupled to the luer component and configured to vent air or fluid from the medication bladder to an external environment, wherein the first needle has a greater length than the second needle, and the second needle is positioned near a vent.

In some aspects, the techniques described herein relate to a dual needle apparatus, wherein the luer component includes external threads.

In some aspects, the techniques described herein relate to a dual needle apparatus, wherein the second needle is shielded by a guard.

In some aspects, the techniques described herein relate to a dual needle apparatus, wherein the first needle and second needle are separated by a distance of at least 2 mm.

In some aspects, the techniques described herein relate to a dual needle apparatus, wherein the apparatus is configured for use with a medication bladder having a flexible inlet seal.

In some aspects, the techniques described herein relate to a dual needle apparatus, wherein the apparatus is configured to be sterilized prior to use.

In some aspects, the techniques described herein relate to a method including: inserting a syringe assembly into an input port of a medication bladder, the syringe assembly including: a dual needle apparatus including a first needle and a second needle, the first needle longer than the second needle; and a reservoir including a quantity of medication; causing at least the quantity of medication to flow from the reservoir to the medication bladder through the first needle, wherein the second needle facilitates a release of pressure caused by the flow of medication through the first needle into the medication bladder at least by facilitating the release of at least one of air or medication to an external environment.

In some aspects, the techniques described herein relate to a method, wherein the medication includes insulin.

In some aspects, the techniques described herein relate to a method, wherein inserting the syringe assembly into the input port of the medication bladder includes inserting the dual needle apparatus through an inlet seal coupled to the input port, wherein after insertion the first needle is in communication with an interior cavity of the medication bladder and the reservoir, and wherein the second needle is in communication with the interior cavity of the medication bladder and with the external environment.

In some aspects, the techniques described herein relate to a method, wherein insulin is released to an external environment through the second needle.

In some aspects, the techniques described herein relate to a method, wherein the released insulin contacts an exposed area of skin of a patient.

In some aspects, the techniques described herein relate to a method, wherein the quantity of medication is greater than a storage capacity of the medication bladder.

In some aspects, the techniques described herein relate to a method, wherein the external environment includes air of a room environment.

In some aspects, the techniques described herein relate to a method, wherein the dual needle apparatus is disposable.

In some aspects, the techniques described herein relate to a method including: depleting air from a syringe assembly, the syringe assembly including: a dual needle apparatus including a first needle and a second needle, the first needle longer than the second needle; and a reservoir configured to hold a quantity of medication; inserting the syringe assembly into a vial, the vial including at least the quantity of medication; and extracting the medication from the vial to the reservoir through the first needle, wherein, when extracting the medication from the vial, the second needle is configured to facilitate movement of the medication from the vial at least by allowing air from an external environment to pass into the vial during extraction, the air creating a pressure against the medication.

In some aspects, the techniques described herein relate to a method, wherein the medication includes insulin.

In some aspects, the techniques described herein relate to a method, wherein the external environment includes air of a room environment.

In some aspects, the techniques described herein relate to a method, wherein inserting the syringe assembly into the vial includes inserting the dual needle apparatus into the vial, wherein after insertion, the first needle is in communication with an interior cavity of the vial and the reservoir.

In some aspects, the techniques described herein relate to a method, wherein the dual needle apparatus is disposable.

In some aspects, the techniques described herein relate to a method, wherein the method further includes sterilizing the dual needle apparatus prior to use.

### BRIEF DESCRIPTION OF DRAWINGS

These and other features, aspects, and advantages of the present application are described with reference to drawings of certain embodiments, which are intended to illustrate, but not limit, the present disclosure. It is to be understood that the attached drawings are for the purpose of illustrating concepts disclosed in the present application and may not be to scale:

FIG. 1A illustrates an example disease management system.

FIG. 1B illustrates an example disease management system that may be part of a disease management environment.

FIG. 2 illustrates a view of an example medication bladder in accordance with some aspects of the disclosure.

FIG. 3 illustrates a view of an example dual needle apparatus in accordance with some aspects of the disclosure.

FIG. 4 illustrates a view of an example dual needle apparatus including an interior of the dual needle apparatus in accordance with some aspects of the disclosure.

FIG. 5A illustrates a view of an example dual needle apparatus and the medication bladder in accordance with some aspects of the disclosure.

FIG. 5B illustrates a view of at least part of the example dual needle apparatus and a portion of the medication bladder, in accordance with some aspects of the disclosure.

FIG. 6 illustrates an example method for delivery of medication to the interior of an example medication bladder.

FIG. 7 illustrates an example method for extraction of medication from a medication vial with an example dual needle apparatus.

### DETAILED DESCRIPTION

Although certain preferred examples are disclosed below, inventive subject matter extends beyond the specifically disclosed examples to other alternative examples and/or uses and to modifications and equivalents thereof. Thus, the scope of the claims that may arise here is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular embodiment. Thus, for example, various examples may be carried out in a manner that achieves or optimizes one advantage or group of advantages as taught herein without necessarily achieving other aspects or advantages as may also be taught or suggested herein.

Certain chemical substances, such as medications for management of diabetes or other physiological conditions, can be administered by a minimally invasive implant, such as a medication delivery pump, or the like. Such systems may utilize medication storage. The medication storage may be configured to hold medication for delivery to the patient. The presence of air in the medication storage may be disadvantageous. The presence of air may, for example, shorten the time the medication storage can be used in delivery of medication to a patient before a refill is required. The presence of air may, as another example, cause an air embolus which could lead to adverse medical consequences for the patient.

Aspects of the present disclosure provide for a dual needle apparatus, which may be used to deliver medication to a medication storage. Medication storage may, in some examples, be part of a minimally invasive implant, such as a medication delivery pump or the like. The dual needle apparatus described herein provides significant advantages over conventional single-needle systems. By enabling simultaneous delivery of medication and venting of air or excess medication, the apparatus reduces the risk of air bubbles, improves filling efficiency, and allows for more accurate dosing.

The dual needle apparatus may include a first needle and a second needle, where the first needle is longer than the second needle. Each needle may have two ends separated by a shaft. An end of the first needle may be in communication with a medication supply, such as medication in a reservoir component of a syringe assembly. An end of the second needle may be in communication with a vent to an external environment.

By way of illustration, when inserted into a medication vial, one end of the first needle may be in communication with an interior of the medication vial and the other end of the first needle may be in communication with an interior of a reservoir of a syringe assembly. In further examples, an end of the second needle may be in communication with a vent to an external environment and the other end of the second needle may be in communication with the interior of the medication vial.

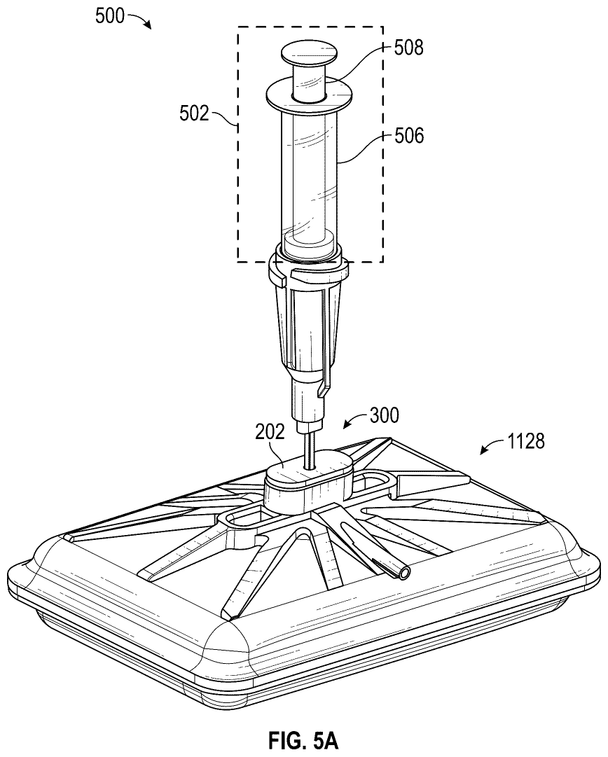

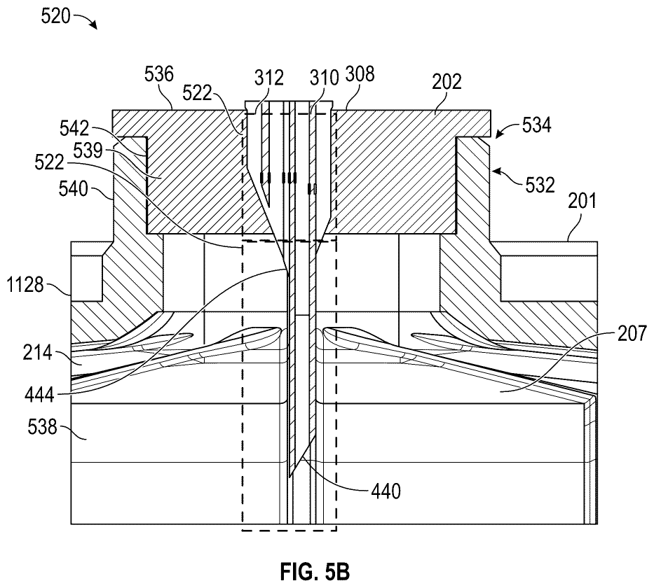

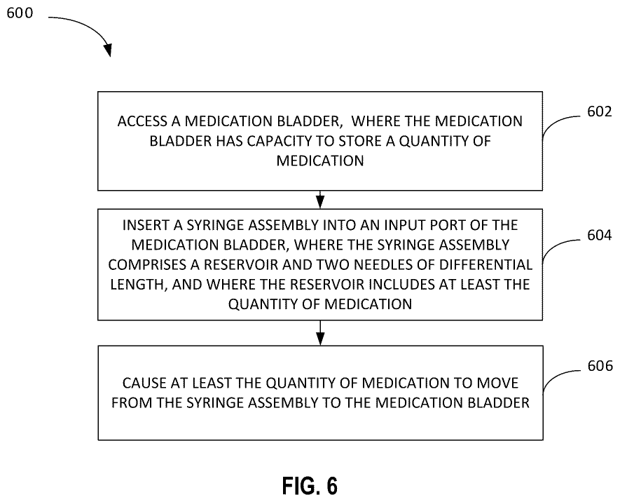

As another illustrative example, when inserted into a medication storage (e.g., for a minimally invasive implant), an end of the first needle may be in communication with a medication supply and the other end of the first needle may be in communication with an interior of the medication storage. For example, the other end of the first needle may be in communication with an interior of a medication bladder of a minimally invasive implant, as will be described with respect to FIGS. 5A-6. In further examples, an end of the second needle may be in communication with a vent to an external environment and the other end of the second needle may be in communication with the interior of the medication bladder.

When delivering medication to the interior of the medication bladder, the second needle may allow at least one of air or medication to vent to the external environment. The medication bladder may for example initially include air, and the second needle may allow this air to leave the medication bladder through the vent as the first needle delivers medication to the interior of the medication bladder. The second needle may, in some examples, allow medication to leave the medication bladder through the vent as the first needle delivers medication to the interior of the medication bladder. A caregiver may interpret the medication leaving the medication bladder through the vent as an indication that the medication bladder is full of medication.

The external environment may be a clinical environment, such as a hospital room, a doctor's office, or the like. However, in some examples, the external environment may be in a patient's home, or the like. Advantageously, because certain medications, such as insulin, are not caustic to the skin, a patient may be able to fill the medication bladder without the need for extra protective equipment which may be difficult for a patient to utilize or implement in a home environment. Thus, the medication may be filled or overfilled such that the medication may enter the patient's environment without harm to the patient during filling or cleanup.

In some examples, the patient may fill the bladder prior to applying the bladder and/or minimally invasive implant to the patient. In some examples, the patient may fill the bladder after applying the bladder and/or minimally invasive implant to the patient. For example, the patient may remain coupled to the minimally invasive implant including the medication bladder during a filling procedure. Accordingly, the medication may, in some examples, leave the medication bladder and contact the patient's skin. If the medication is not caustic, such as if the medication is insulin, the patient may advantageously remain in the external environment as the medication bladder is filled.

The above-described aspects and other aspects of the disclosure will now be described with regard to certain examples, embodiments, and aspects, which are intended to illustrate, but not limit, the disclosure. The foregoing aspects and many of the attendant advantages of this disclosure will become more readily appreciated as the same become better understood by reference to the following description, when taken in conjunction with the accompanying drawings.

### Example Disease Management System

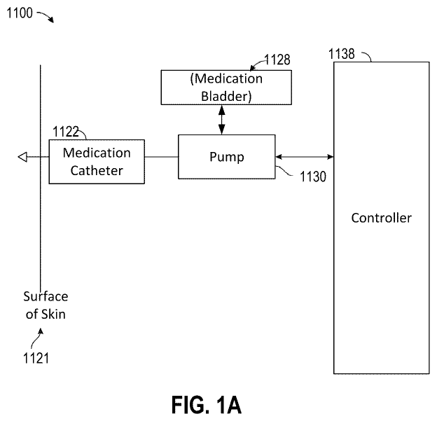

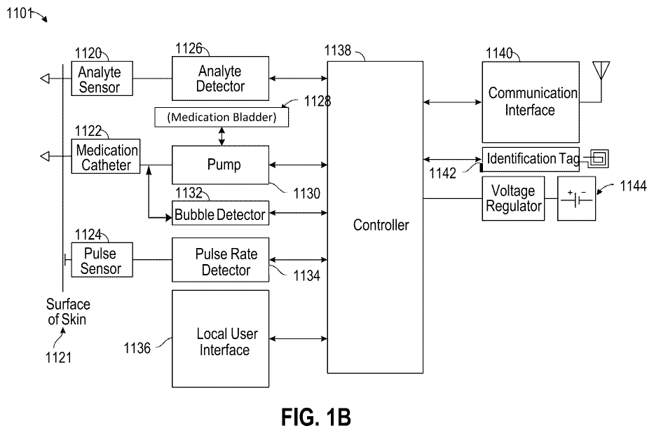

FIG. 1A illustrates an example disease management system 1100. The disease management system 1100 may be configured to deliver medication (e.g., as insulin, glucagon, another medication, some combination thereof, etc.). An example disease management system 1100 and/or features thereof is described with respect to U.S. Pat. Pub. No. 2023/0115397, filed Aug. 4, 2022, titled “MEDICATION DELIVERY PUMP FOR REDUNDANT STAGGERED GLUCOSE SENSOR INSULIN DOSAGE SYSTEM,” which is incorporated by reference herein in its entirety.

The disease management system 1100 may, in addition to medication, also administer other chemical substances, such as various additives, for patient treatment. As used herein, additives may refer to chemical substances used to impact performance of the medication. For example, additives may impact how the medication is absorbed in a manner that impacts the time it takes for the medication to take effect. Additives may also impact how long the medication is viable. Additives may include, but are not limited to preservatives, stabilizers, surfactants, buffers, antimicrobials, zinc, another chemical substance, or some combination thereof. Example chemical substances included in the medication are described and/or illustrated in U.S. Pat. Pub. No. 2022/0096603 filed Sep. 28, 2021, titled “INSULIN FORMULATIONS AND USES IN INFUSION DEVICES,” which is incorporated by reference herein in its entirety. As used herein, systems and methods describing storing, movement, and/or administration of medication may also be used to administer medication in addition to other chemical substances, such as various additives.

The example disease management system 1100 includes a medication catheter 1122, a medication bladder 1128, a pump 1130, and a controller 1138. The controller 1138 may control components of example disease management system 1100, such as medication catheter 1122, a medication bladder 1128, and a pump 1130. The controller 1138 may include one or more hardware processors, such as a printed circuit board (PCB), or the like. The controller 1138 may, in some examples, calculate a dose based on physiological parameter data for a patient. The controller 1138 may alternatively receive a calculated dose value and administer medication, such as insulin, by actuation of an actuated pump, such as pump 1130. The controller 1138 may record device activity and transfer the recorded data to non-volatile secure memory space. In some examples, a person (e.g., caregiver, a patient, or the like) may determine to no longer use the disease management system 1100. For example, the person may want to replace the disease management system 1100. In further examples, the controller 1138 can be configured to lock operation, and create a data recovery module to permit authenticated access to the recorded data if needed.

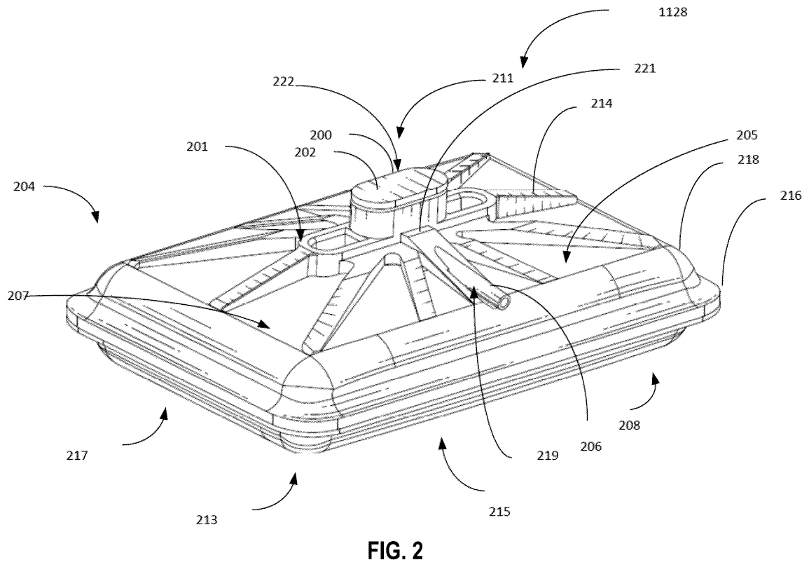

The medication bladder 1128 may include any of the components and features described with respect to medication bladders described and/or illustrated in U.S. patent application Ser. No. 18/419,408 filed Jan. 22, 2024, titled “MEDICATION BLADDER FOR MEDICATION STORAGE,” which is incorporated by reference herein in its entirety. The medication bladder 1128 may, for example, be configured to hold and/or release medication to be administered. The medication bladder 1128 may be configured to hold the medication for a prolonged period, such as 1 day, 3 days, 6 days, or more. The time period for storage may impact the size of the medication bladder 1128. The disease management system 1100 may, in some examples, include a plurality of medication bladders 1128 as reservoirs of medications. Each medication bladder 1128 may, in further examples, store a different composition of medication, additives, and the like, or some combination thereof. In a non-limiting example, at least one medication bladder 1128 may include insulin. While the description above describes the medication bladder 1128 as part of disease management system 1100, other examples are also possible. Illustratively, the medication bladder 1128 may be included in a standard insulin pump. The medication bladder may be removeable from the standard insulin pump using any of the methods described herein. Components and features of the medication bladder 1128 will be described in more detail with respect to FIG. 1B and FIG. 2.

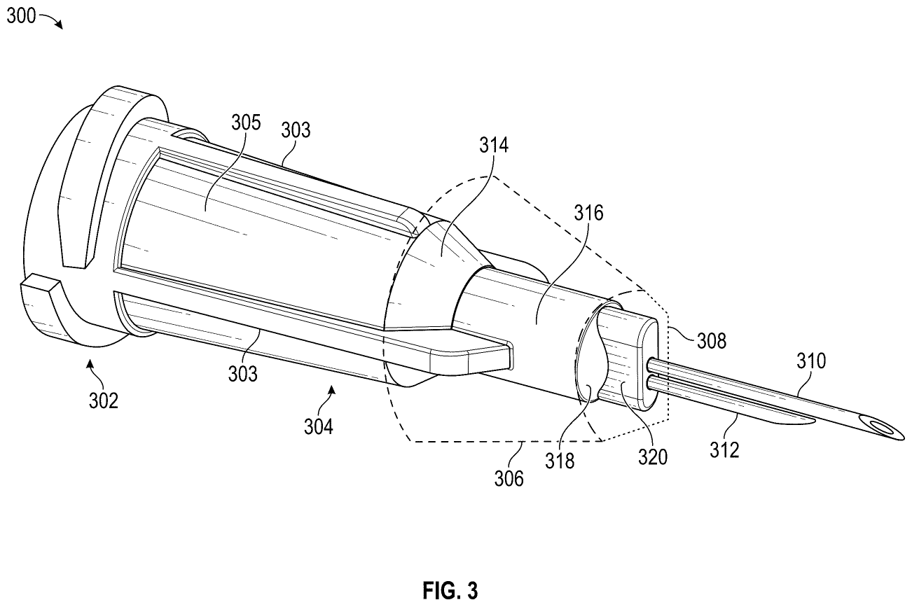

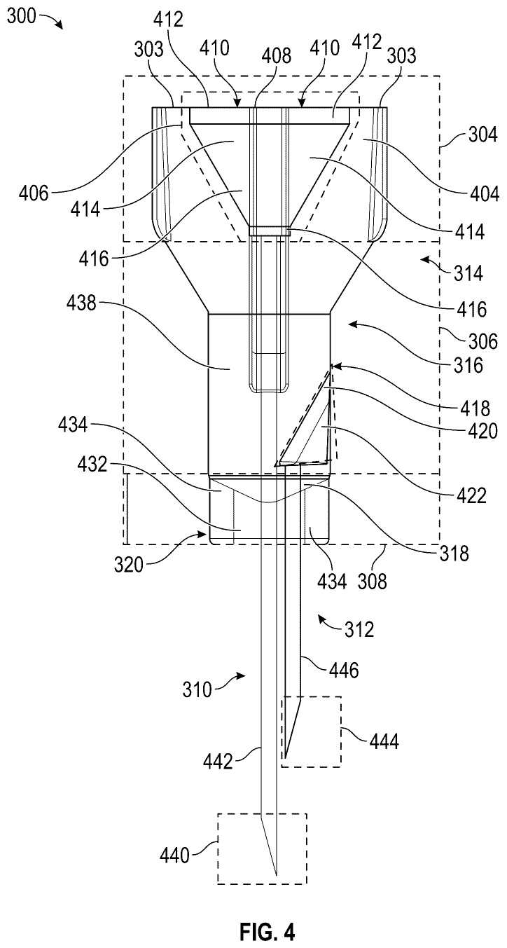

A caregiver, when filling a medication bladder 1128 may utilize a syringe assembly (e.g., syringe assembly 502 of FIG. 5A) comprising a dual needle apparatus 300, as will be described herein with respect to FIGS. 3-7. The dual needle apparatus 300 may, for example, include at least two needles of differential length, such as first needle 310 and second needle 312 of FIG. 3. First needle 310, as illustrated in FIG. 3, is longer than a second needle 312 of the two needles. An end of the first needle 310 may be in communication with a quantity of medication. An end of the second needle 312 may be in communication with a vent to an external environment (e.g., a hospital room, a doctor's office, a patient's home, etc.). Components of the dual needle apparatus 300 will be described further with FIGS. 3-4. Example use of the dual needle apparatus 300 to fill a syringe assembly 502 will be described further with respect to FIG. 5A. Example use of the dual needle apparatus 300 to fill a medication bladder, such as medication bladder 1128, is described with respect to FIGS. 5A-6.

A disease management system 1100 may, in some examples, include a medication catheter 1122. The medication catheter 1122 may be configured to administer medication to the patient. For example, the pump 1130 may apply a force (e.g., pressure, vacuum force, etc.) to medication bladder 1128 causing medication to flow from medication bladder 1128 to medication catheter 1122. The medication may, for example, flow from medication bladder 1128 to pump 1130 to medication catheter 1122.

The system may operate on battery power and support both shelf-life and reliable operation once applied to the patient. Battery life may be managed through control of several planned levels of sleep and power consumption. To support this reliability, a controller can monitor several system-health parameters, and monitor temperatures of the included medication, and ambient temperature for the life of the device.

Any of the components of disease management system 1100 may be separable from other components of the disease management system 1100. Illustratively, medication bladder 1128 may be separated from the disease management system 1100. In further examples, medication bladder 1128 may be refilled subsequent to separation from the disease management system 1100. Medication bladder 1128 may additionally, or alternatively be cleaned subsequent to separation from disease management system 1100.

FIG. 1B shows a block diagram of an example disease management system 1101, that may be in accordance with some aspects of the present disclosure. An example disease management system 1101 and/or features thereof is described with respect to U.S. Pat. Pub. No. 2021/0236729, filed Jan. 28, 2021, titled “REDUNDANT STAGGERED GLUCOSE DISEASE MANAGEMENT SYSTEM,” U.S. patent application Ser. No. 18/605,630 filed Mar. 14, 2024, titled “MODULAR DISEASE MANAGEMENT DEVICE AND AUTOMATED NEEDLE AND CANNULA INSERTION DEVICE,” U.S. Pat. Pub. No. 2023/0115397, filed Aug. 4, 2022, titled “MEDICATION DELIVERY PUMP FOR REDUNDANT STAGGERED GLUCOSE SENSOR INSULIN DOSAGE SYSTEM,” each of which are incorporated by reference herein in their entirety.

In some examples, the disease management system 1101 may be part of a disease management environment. A disease management system 1101 may be configured to measure one or more physiological parameters of a patient, such as pulse, skin temperature, or other values. The disease management system 1101 may further be configured to measure one or more analytes present in the blood of a patient, such as glucose, lipids, or other analytes. The disease management system 1101 may, in some examples, administer medication (e.g., as insulin, glucagon, another medication, some combination thereof, etc.). The disease management system 1101 may, in addition to medication, also administer other chemical substances, such as various additives, for patient treatment. As used herein, additives may refer to chemical substances used to impact performance of the medication. For example, additives may impact how the medication is absorbed in a manner that impacts the time it takes for the medication to take effect. Additives may also impact how long the medication is viable. Additives may include, but are not limited to preservatives, stabilizers, surfactants, buffers, antimicrobials, zinc, another chemical substance, or some combination thereof. Example chemical substances included in the medication are described and/or illustrated in U.S. Pat. Pub. No. 2022/0096603 filed Sep. 28, 2021, titled “INSULIN FORMULATIONS AND USES IN INFUSION DEVICES,” which is incorporated by reference herein in its entirety. As used herein, systems and methods describing storing, movement, and/or administration of medication may also be used to administer medication in addition to other chemical substances, such as various additives.

The disease management system 1101, in some examples, may be modular. As used herein, “modular” may mean that at least some of the components of the disease management system 1101 may be separable from other components of the disease management system 1101. Disease management system 1101 may include any of the components or features described with respect to modular disease management systems described and/or illustrated in U.S. patent application Ser. No. 18/605,630 filed Mar. 14, 2024, titled “MODULAR DISEASE MANAGEMENT DEVICE AND AUTOMATED NEEDLE AND CANNULA INSERTION DEVICE,” which is incorporated by reference herein in its entirety.

By way of illustration, the disease management system 1101 may be configured to supply power, as needed, to the components illustrated in FIG. 1B. At least some of the components illustrated in FIG. 1B may be modules removably coupled to the disease management system 1101 through one or more electrical connectors, not shown. For example, the controller 1138 may be a reusable module. When a patient needs to replace the disease management system 1101, the patient can remove the controller 1138 from a first disease management system 1101 and couple the controller module 1138 to a second disease management system 1101. The controller 1138 may then control components of the second disease management system 1101. As another example, the controller 1138 of the first disease management system 1101 may malfunction, and a new controller 1138 may be placed in the first disease management system 1101 by a patient or caregiver. The new controller 1138 may then control components of the first disease management system 1101.

Other components of the disease management system 1101 may also be modules removably coupled to the disease management system 1101. For example, the medication bladder 1128 may be replaceable and/or removable from the disease management system 1101. When removed from the disease management system 1101, the medication bladder 1128 may be renewed (such as cleaned and/or refilled with medication) and/or a new medication bladder 1128 may be inserted in place of the medication bladder 1128 into the disease management system 1101. The medication bladder 1128 may alternatively be refilled while installed into the disease management system 1101.

The medication bladder 1128 may include any of the components and features described with respect to FIG. 1A. The medication bladder 1128 may additionally, or alternatively, include any of the components or features of medication bladders described and/or illustrated in U.S. patent application Ser. No. 18/419,408 filed Jan. 22, 2024, titled “MEDICATION BLADDER FOR MEDICATION STORAGE,” which is incorporated by reference herein in its entirety. While FIG. 1B describes the medication bladder 1128 as part of a disease management system 1101, other examples are also possible. Illustratively, the medication bladder 1128 may be included in a standard insulin pump. The medication bladder may be removeable from the standard insulin pump using any of the methods described above with respect to the disease management system 1101. Components and features of the medication bladder 1128 will be described in more detail with respect to FIG. 2.

Using any of the methods described with respect to FIG. 1A, a person (e.g., caregiver, a patient, or the like) when filling a medication bladder 1128 may utilize a syringe assembly (e.g., syringe assembly 502 of FIG. 5A) comprising a dual needle apparatus 300, as will be further described herein with respect to FIGS. 3-7.

A disease management system 1101 may, in some examples, include a medication catheter 1122. The medication catheter 1122 may be configured to administer medication to the patient. For example, the pump 1130 may apply a force (e.g., pressure, vacuum force, etc.) to medication bladder 1128 causing medication to flow from medication bladder 1128 to medication catheter 1122. The medication may, for example, flow from medication bladder 1128 to pump 1130 to medication catheter 1122.

In some examples, a disease management system 1101 may be configured to communicate with one or more hardware processors that may be external to the disease management system 1101, such as a cloud-based processor or user device. A disease management system 1101 may include an identification tag 1142, such as a near field communication tag or a radio frequency identification (RFID) tag to support authentication and pairing with a user device (for example, smart phone or smart watch), a communication interface 1140. The communication interface 1140 may support Bluetooth communication with additional disease management systems or devices, and Bluetooth communication with a paired user device running an associated control application. The communication interface 1140 may also support communication with other communication protocols including, but not limited to, Transmission Control Protocol/Internet Protocol (TCP/IP), Message Queuing Telemetry Transport (MQTT), or Wi-Fi.

To support ease of use and safe interaction with the patient, the system may incorporate user input through a tap-detecting accelerometer and provide feedback via an audio speaker, haptic vibration, and/or optical indicators. The system may operate on battery power and support both shelf-life and reliable operation once applied to the patient. Battery life may be managed through control of several planned levels of sleep and power consumption. To support this reliability, a controller (e.g., controller 1138) can monitor several system-health parameters, and monitor temperatures of the included medication, and ambient temperature for the life of the device.

As illustrated in FIG. 1B, a controller 1138 of the disease management system 1101 may be configured to communicate and control one or more components of the disease management system 1101. The controller 1138 may include one or more hardware processors, such as a printed circuit board (PCB), or the like. The controller 1138 may be configured to communicate with peripheral devices or components to support the accurate measurement of physiological parameters and blood analytes, such as patient pulse, temperature, and blood glucose, using detector electronics. The controller 1138 may subsequently calculate dose or receive a calculated dose value and administer medication, such as insulin, by actuation of an actuated pump. The controller 1138 may record device activity and transfer the recorded data to non-volatile secure memory space. In some examples, a user (e.g., a manufacturer, caregiver, or patient) may determine to no longer use the disease management system 1101. For example, the user may want to replace the disease management system 1101. In further examples, the controller 1138 can be configured to lock operation, and create a data recovery module to permit authenticated access to the recorded data if needed.

In some examples, a disease management system 1101 may be configured to include a plurality of physiological sensors, such as a physiological sensor 1124. The physiological sensor 1124 may include a pulse rate sensor, temperature sensor, pulse oximeter, and the like, or a combination thereof. Each physiological sensor 1124 may be configured to communicate with a physiological detector 1134. The physiological detector 1134 may be configured to receive a signal or signals of the physiological sensor 1124. The physiological detector 1134 may also be configured to measure or determine a physiological value from the signal. Determinations and measurements by the physiological detector 1134 may be responsive to signals from the controller 1138. The physiological detector 1134 may additionally, or alternatively, communicate the physiological values to a destination, such as controller 1138.

A disease management system 1101 may include an analyte sensor 1120. The analyte sensor 1120 may be configured to detect analytes in the patient's blood. For example, an analyte sensor 1120 can include a glucose sensing probe configured to pierce the surface of the skin 1121. In some examples, a disease management system 1101 may include a plurality of analyte sensors 1120 to detect one or more analytes. In some examples, an analyte sensor 1120 may be configured to detect a plurality of analytes. Sensed analytes may include, but are not limited to, glucose, insulin, and other analytes. An analyte sensor 1120 may be configured to communicate with an analyte detector 1126. The analyte detector 1126 may be configured to receive a signal of one or more analyte sensors 1120 in order to measure one or more analytes in the blood of the patient. The analyte detector 1126 may be configured to communicate with the controller 1138. For example, the analyte detector 1126 may be configured to, for example, send analyte values to the controller 1138 and receive control signals from the controller.

A disease management system 1101 may, in some examples, include one or more local user interfacing components 1136. For example, a local user interfacing component 1136 may include, but is not limited to, one or more optical displays, haptic motors, audio speakers, and user input detectors. In some examples, an optical display may include an LED light configured to display a plurality of colors. In some examples, an optical display may include a digital display of information associated with the disease management system 1101, including, but not limited to, device status, medication status, patient status, measured analyte or physiological values, and the like, or a combination thereof. In some examples, a user input detector may include an inertial measurement unit, tap detector, touch display, or other component configured to accept and receive user input. In some examples, audio speakers may be configured to communicate audible alarms related to device status, medication status, user status, and the like, or a combination thereof. A controller 1138 may be configured to communicate with the one or more local interfacing components 1136 by, for example, receiving user input from the one or more user input components or sending control signals to, for example, activate a haptic motor, generate an output to the optical display, generate an audible output, or otherwise control one or more of the local user interfacing components 1136.

A disease management system 1101 may include a communication interface 1140 with one or more communication components. A communication component can include, but is not limited to, one or more radios configured to emit Bluetooth, cellular, Wi-Fi, or other wireless signals. In some examples, a communication component can include a port for a wired connection. Additionally, a disease management system 1101 may include an identification tag 1142 to facilitate in communicating with one or more hardware processors. The one or more communication components of the communication interface 1140 and identification tag 1142 may be configured to communicate with the controller 1138 in order to send and/or receive information associated with the disease management system 1101. For example, a controller 1138 may communicate medication information and measured values through the one or more components of communication interface 1140 to an external device. Additionally, the controller 1138 may receive instructions associated with measurement sampling rates, medication delivery, or other information associated with operation of the management system 1101 through the one or more components of communication interface 1140 from one or more external devices.

A disease management system 1101 may include one or more power components 1144. The power components 1144 may include, but are not limited to, one or more batteries and power management components, such as a voltage regulator. Power from the one or more power components 1144 may be accessed by the controller 1138 and/or other components of the disease management system 1101 to operate the disease management system 1101.

A disease management system 1101 may have one or more power and sleep modes to help regulate power usage. For example, a disease management system 1101 may have a sleep mode. The sleep mode may be a very low power mode with minimal functions, such as the RTC (or real time clock) and alarms to wake the system and take a temperature measurement of the system, or the like. In another example, a disease management system 1101 may include a measure temperature mode which may correspond to a low power mode with reduced functions. The measure temperature mode may be triggered by the RTC where the system is configured to take a temperature measurement, save the value, and return the system to a sleep mode. In another example, a disease management system 1101 may include a wake up mode. The wake up mode may be triggered by an NFC device and allow the system to pair with an external device with, for example, Bluetooth. If a pairing event does not occur, the system may return to sleep mode. In another example, a disease management system 1101 may include a pairing mode. The pairing mode may be triggered by an NFC device. When a controlling application is recognized, the system may proceed to pair with the application and set the system to an on condition and communicate to the cloud or other external device to establish initial data movement. In another example, a disease management system 1101 may include a rest mode where the system is configured to enter a lower power mode between measurements. In another example, a disease management system 1101 may include a data acquisition mode where the system is configured to enter a medium power mode where data acquisition takes place. In another example, a disease management system 1101 may include a parameter calculation mode where the system is configured to enter a medium power mode where parameter calculations, such as a blood glucose calculation, are performed and data is communicated to an external device and/or the cloud. In another example, a disease management system 1101 may include a pump mode where the system is configured to enter a higher power mode where the pump draws power to deliver medication to the patient.

In some examples, a disease management system 1101 may include one or more connector test points 1146. The connecter test points may be configured to aid in programming, debugging, testing or other accessing of the disease management system 1101. In some examples, connector test points 1146 may include, for example, a GPIO spare, UART receiver or transmitter, and the like, or a combination thereof.

### Example Medication Bladder

FIG. 2 illustrates an example of an exterior side of a rigid portion 204 of an example medication bladder 1128 that may be part of a medication pump system, such as disease management system 1100 of FIG. 1A. The medication bladder 1128 may alternatively be part of an example disease management system 1101 of FIG. 1B. The medication bladder 1128 may, in some examples, include the features described above with respect to medication bladder 1128 of FIGS. 1A-1B in addition to features described below. However, other implementations in other medication delivery and/or storage systems are also possible. As one example, instead of a rigid portion 204 and flexible portion 208, the medication bladder 1128 may comprise two flexible portions coupled to form an enclosure.

In some implementations, such as in the illustrated example of FIG. 2, the medication bladder 1128 may include at least two exterior housing components configured to at least partially couple. The exterior housing components, when coupled, may be configured to form enclosure (e.g., a bladder, pouch, etc.) within the medication bladder 1128 for holding medication, and the like, or some combination thereof, for administration to a wearer of a medication management system, including, but not limited to, a disease management system 1100 such as described with reference to FIG. 1A, a disease management system 1101 such as described with reference to FIG. 1B or another medication delivery system. The medication may, in further examples, be fluids.

A dual needle apparatus 300, as described herein, may be used in filling the medication bladder 1128 with medication, such as insulin, or the like. The dual needle apparatus 300 may include one or more needles of differential length, such as first needle 310 of FIG. 3 and second needle 312 of FIG. 3. First needle 310 may be longer than the second needle 312. The second needle 312 may advantageously facilitate filling of the medication bladder 1128 by allowing at least one of medication or air to vent to an external environment (e.g., such as a hospital room, a doctor's office, or the like). Components of the dual needle apparatus 300 will be described with respect to FIGS. 3-4. Use of the dual needle apparatus 300 for filling the medication bladder 1128 will be described with respect to FIGS. 5A-6.

The at least two exterior housing components may include at least one rigid portion 204 and at least one flexible portion 208. The at least one rigid portion 204 may be configured to maintain shape under a threshold amount of pressure and the at least one flexible portion 208 may be configured to move, change shape, deflate, or otherwise modify its shape or orientation when exposed to at least a minimum threshold positive or negative pressure. The material of the rigid portion 204 may be a rigid material including, but not limited to, ABS, polypropylene (PP), polycarbonate (PC) or metal, or some combination thereof. The material of the flexible portion 208 may be a soft material including, but not limited to, thermoplastic elastomer (TPE), silicone, or some combination thereof. The flexible portion 208 may be in the form of a soft film of the selected material or materials. Portions of the rigid portion 204 and flexible portion 208 may be coated with an anti-aggregation coating including, but not limited to, silicone dioxide, low-density polyethylene (LDPE) coating, LDPE film, or zinc oxide.

In some examples, the at least one rigid portion 204 may have at least one input port 200 with at least one inlet and at least one output port 206. The at least one rigid portion 204 may include a geometry having at least one external surface 205, at least one internal surface 405, at least one wall 218, and at least one lip or edge 216. The geometry of the rigid portion 204 may be or include guiding structures such as curved surfaces or inclined planes, which may facilitate the movement of medication to the output port or output ports 206.

As shown in FIG. 2, the exterior surface 205 of the rigid portion 204 may be on an exterior side of the enclosure formed by rigid portion 204 and flexible portion 208. The at least one external surface 205 of the rigid portion 204 may include at least one top surface 207, and at least one structure 214. The top surface 207 may form an approximately flat surface. The top surface 207 may additionally, or alternatively, form an inclined surface. The inclined surface may tilt from an edge, such as edge 216, towards a central region 211.

In some examples, the at least one structure 214 may be configured to extrude, couple to, or otherwise extend from the at least one top surface 207. The at least one structure 214 may be configured to provide a supporting structure for the rigid portion 204, improving the strength and/or rigidity of the rigid portion 204. In some examples, the at least one structure 214 may be configured to support one or more areas of the rigid portion 204 associated with at least one channel on an interior of the rigid portion 204.

In some examples, the at least one structure 214 may include a plurality of structures, such as 6, 8, 10, or more structures or structural components. The structures 214 may protrude from the top surface 207. The at least one structure 214 may be configured to extend from at least a portion of an edge 216 or wall 218 of the rigid portion 204 towards a central region 211 of the rigid portion 204, which may include an input port 200 or other structure 201 surrounding and/or supporting an input port 200, which may or may not form part of the at least one structure 214.

The at least one structure 214 may vary in size and/or shape across the geometry of the rigid portion 204. For example, the at least one structure 214 may be configured to increase in height and/or change shape or profile between an edge 216 and/or wall 218 towards the central region 211. In some examples, the at least one structure 214 may be configured to have a different shape and/or more or fewer structures may be placed on the area of the rigid portion 204 based on a location of the rigid portion 204, such as, where the rigid portion 204 has an approximately rectangular top-down profile, nearer a corner 213, major edge 215, or minor edge 217.

A central region 211 may include at least one structure configured to extend perpendicularly from a major plane of the at least one external surface 205 of the rigid portion 204. Advantageously, the form of the central region 211 may facilitate gathering and/or flow of medication from an interior of a medication bladder 1128 towards an output region 219.

With continued reference to FIG. 2, the central region 211 may include an input region 222 and/or an output region 219. The input region 222 may include an input port 200 and/or associated inlet seal 202. The input port 200 may be configured to have an opening at a center of the central region 211 such that the input port 200 can be accessed from a top of the rigid portion 204. The inlet seal 202 may be configured to at least partially seal the input port 200, as discussed herein.

The central region 211 may further include, in some implementations, at least a part of an output region 219, such as a lateral or otherwise oriented opening 221 of an output region 219. In some examples, the output region 219 may be offset from the central region 211 of the rigid portion 204. In some examples, the output region 219 may be oriented to output medication at a lateral location, such as at, near, or above an edge 216. In some examples, the output region 219 may be configured to extend from a central region 211 towards an edge 216 of the rigid portion 204. However, other configurations are also possible. Additionally, or alternatively, the output region 219 could be located on a flexible portion 208 of the medication bladder 1128. The output region 219 may additionally, or alternatively, protrude upward from the at least one top surface 207 and/or bulk or planar portion of the rigid portion 204, such as illustrated in in FIG. 2.

The output region 219 may be configured to facilitate evacuating medication from the medication bladder 1128. The one or more output ports 206 may serve as outlets to allow medication to exit the medication bladder 1128. For example, the output region 219 may include one or more output ports 206. An output port 206 may facilitate the flow of medication by providing an opening through which the medication can leave the output region 219 and the medication bladder 1128. At least some of output ports 206 may, in some examples, be angled with respect to a horizontal plane aligned with rigid portion 204. The one or more output ports 206 may include, but are not limited to, a tube or cannula extending from a portion of the rigid portion 204, such as a lateral or otherwise oriented opening 221 in a central region 211 of the rigid portion 204. The one or more output ports 206 may be composed of one or more materials, which may include, but is not limited to, metal or silicone. In addition, the one or more output ports may contribute to facilitating the flow of medication through the output region 219 and out of the medication bladder 1128.

In some examples, one or more output ports 206 may be used together to increase the rate of the flow of medication through the output region 219 and out of the medication bladder 1128. In some examples, additional structures (e.g., channels, other types of protrusions, another structure, or some combination thereof) may be used to direct the flow of medication through the one or more output ports to further facilitate the flow of medication through the output region 219 and out of the medication bladder 1128. In some examples, a plurality of output ports 206 of the one or more output ports may be used to direct the medication to different locations. As an example, a plurality of output ports 206 of the one or more output ports 206 may direct the medication to different injection points (e.g., locations on a patient's body configured for receipt of medication, such as by insertion of a needle). In some examples, at least one of the plurality of output ports 206 may be configured to output medication from the medication bladder to a cannula or other channel, such as a cannula 1164, described with reference to FIG. 1B. For example, an output port 206 may be configured to couple to a cannula or other channel that may pass through or be engaged with a pump configured to cause a flow of medication from the medication bladder 1128 towards an injection point on the patient. The coupling of the cannula and output port 206 may be a press fit or other connection type. In other examples, a cannula may be directly coupled to an output region 219 of the rigid portion 204.

Additionally, or alternatively, in examples utilizing a plurality of medication bladders 1128 or a medication bladder 1128 and at least one secondary storage location, a plurality of output ports 206 of the one or more output ports 206 may be used to connect to additional medication bladders or storage locations (e.g., connect an output port 206 of one medication bladder 1128 to the input port 200 of another.) In further examples, the one or more output ports may be used to move medication between one or more medication bladders 1128 or other storage locations. For example, medication may be moved between the one or more medication bladders of a medication delivery system in order to deliver the medication to the patient as determined by a physician and/or by the medication delivery system. Advantageously, moving medication between storage locations, such as a medication bladder 1128, may facilitate mixing or combining of medications or other substances prior to delivery to a patient.

As discussed, the rigid portion 204 may include an input region 222. The input region 222 may include an input or input port 200. The input region 222 may be located at least in part at a central region 211 of the rigid portion. In some examples, the input region 222 may be offset from the center. However, other configurations are also possible. For example, the input region 222 may be located at an edge 516 of the rigid portion 204. Additionally, or alternatively, the input region 222 could be located on a flexible portion 208 of the medication bladder 1128. The input region 222 may additionally, or alternatively, protrude upward from a bulk or planar portion of the rigid portion 204, such as shown in FIG. 2.

In some examples, the input region 222 may include one or more input ports 200. The input port 200 may serve as an inlet into the medication bladder 1128. An inlet seal 202 may be coupled to the input port 200. The input port 200 may be configured to receive the inlet seal 202. The inlet seal 202 might be configured to allow a fill device, such as a needle, to pass through it to fill the medication bladder 1128 with medication. The inlet seal 202 may be able to self-close after the fill device is removed. The material of the inlet seal 202 may be a soft material, such as silicone, which may comprise sealable opening and/or be configured to receive the inlet seal 202, as described above with regards to the input port 200. In some examples, the plurality of input ports 200 may be used together to increase the rate of the flow of medication through the input region 222 and into the medication bladder 1128. In some examples, additional structures (e.g., channels, other types of protrusions, another structure, a needle or needles, another fill device, or some combination thereof) may be used to help direct the flow of medication through the one or more input ports 200 to further facilitate the flow of medication through the input region 222 and into the medication bladder 1128.

### Example Dual Needle Apparatus

FIG. 3 illustrates a view of an example dual needle apparatus 300 in accordance with some aspects of the disclosure. The dual needle apparatus 300 may include a luer component 302, a body component 304, a major needle attachment hub 306, a minor needle attachment hub 308, a first needle 310, and a second needle 312. The dual needle apparatus 300 may include more (or fewer) elements than those shown in FIG. 3. It is not necessary, however, that all of these elements be present, and some elements may be different than shown.

Luer component 302 includes a cylindrical base. However, in some examples, luer component 302 may be tapered such that the end distal from body component 304 has a larger radius than the end proximate to the body component 304. Luer component 302 includes one or more external threads protruding from an exterior surface of the cylindrical base. The external threads may correspond to cavities along the interior surface of the luer component 302. The interior surface of the luer component 302 may alternatively be substantially flat. For example, the interior surface of luer component 302 may be cylindrical. Alternatively, the interior surface of the luer component 302 may be tapered such that the end distal from body component 304 has a larger radius than the end proximate to the body component 304. The luer component 302 may, in some examples, include polypropylene (PP) plastic.

The external threads of luer component 302 may facilitate coupling of the dual needle apparatus 300 with an external system. For example, luer component 302 may be a female luer component intended to facilitate a luer lock connection. Illustratively, the luer component 302 may facilitate coupling of the dual needle apparatus 300 to a standard syringe assembly. The volume of the syringe assembly selected may vary, such as based on the volume of the medication bladder 1128. As one example, if the volume of the medication bladder 1128 is 2.5 mL the volume of the syringe assembly (e.g., syringe assembly 502) may be around 2.5 mL. However, if the medication bladder 1128 is a larger or smaller, the volume of the syringe assembly 502 may be correspondingly larger or smaller.

The luer component 302 may facilitate coupling with syringe assembly 502 of FIG. 5A. The syringe assembly 502 may, for example, include a male luer component to facilitate connection with the luer component 302. By way of illustration, a person (e.g., a caregiver, patient, etc.) may couple the dual needle apparatus 300 to the syringe assembly 502. During the coupling process, the caregiver may align a longitudinal axis of the dual needle apparatus 300 with a longitudinal axis of syringe assembly 502. The caregiver may subsequently bring the luer component 302 closer to a luer component of the syringe assembly 502. The person may, for example, bring the luer component 302 into contact with a luer component of the syringe assembly 502. The luer component of the syringe assembly 502 may be configured to accept the luer component 302. For example, the interior surface of the luer component of the syringe assembly 502 may include cavities forming internal threads. The person may rotate the dual needle apparatus 300 such that the external threads of luer component 302 slide or otherwise move along the internal threads of the luer component of the syringe assembly 502 until a stopping point is reached. The stopping point may be, for example, a point at which an end of the external threads of luer component 302 has met an end of the internal threads of the luer component of the syringe assembly 502. Once the stopping point has been reached the dual needle apparatus 300 may be coupled to the syringe assembly 502 to form a luer lock fitting. Coupling using the luer component 302 advantageously allows for increased security of the coupling at least by reducing or eliminating potential leakage of medication. Use of the dual needle apparatus 300 with syringe assembly 502 will be described in more detail herein with respect to FIGS. 5A-7.

While the example above describes a luer component 302 with threads, other alternatives are possible. As another example, luer component 302 may not include threads. The luer component 302 may instead be configured to form a luer slip fitting with a luer component of syringe assembly 502. A caregiver may, for example, apply a force to slide the luer component 302 along an interior surface of the luer component of syringe assembly 502 until a stopping point is reached. Use of the dual needle apparatus 300 with syringe assembly 502 will be described in more detail herein with respect to FIGS. 5A-7.

In some embodiments, the dual needle apparatus may include a connector other than a luer lock fitting. For example, the connector may be a slip-tip fitting, a threaded fitting, a bayonet fitting, or any other standardized connector suitable for coupling to a syringe assembly or medication reservoir. The selection of connector type may depend on the intended application, compatibility with existing medical devices, and user preference.

The needles of the dual needle apparatus may be fabricated from a variety of materials. In certain embodiments, the needles are made of stainless steel, which provides strength, corrosion resistance, and biocompatibility. Alternatively, the needles may be formed from titanium, which offers reduced weight and enhanced biocompatibility, or from polymer composites, which may be selected for cost, flexibility, or specific chemical compatibility with the medication. The choice of material may be determined by the requirements of the medication, the environment of use, and manufacturing considerations.

Turning to body component 304, as illustrated in FIG. 3, body component 304 may be cylindrical such that the end of the body component 304 distal from major needle attachment hub 306 has a diameter substantially similar to the end of body component 304 proximate to major needle attachment hub 306. Alternatively, body component 304 may be tapered such that the end distal from major needle attachment hub 306 has a larger diameter than the end proximate to the major needle attachment hub 306.

The body component 304 includes one or more flanges 303 protruding from an exterior surface of body component 304. Some of the one or more flanges 303 may additionally protrude from the surface of major needle attachment hub 306. However, at least some of the flanges 303 may extend across body component 304 alone. The flanges 303 may advantageously provide support for the dual needle apparatus 300. The flanges may, for example, facilitate maintenance of the shape of dual needle apparatus 300 as medication flows through dual needle apparatus 300. The flanges 303 may, in some examples, correspond to cavities on an interior surface of body component 304 and/or the interior portion of major needle attachment hub 306. Alternatively, the interior of body component 304 may be substantially flat. The interior portion of major needle attachment hub 306 may also be substantially flat with respect to the location of the flanges on an exterior surface of the major needle attachment hub 306. An interior of major needle attachment hub, such as major needle attachment hub 306, will be described in more detail with respect to FIG. 4.

The major needle attachment hub 306 may include a first portion 314 and a second portion 316. The first portion 314 is tapered such that an end of the first portion 314 proximate to the body component 304 has a diameter substantially similar to the body component 304. Additionally, as illustrated in FIG. 3, an end of the first portion 314 proximate to the body component 304 has a larger diameter than an end of first portion 314 distal to body component 304. Alternatively, in some examples, first portion 314 may be cylindrical such that both ends of first portion 314 have substantially the same diameter. Second portion 316, as illustrated in FIG. 3, is cylindrical such that both ends of second portion 316 have substantially the same diameter. However, in some examples, the second portion 316 may be tapered such that an end proximate to minor needle attachment hub 308 has a smaller diameter than an end of second portion 316 distal to minor needle attachment hub 308. The major needle hub 306 may, in some examples, include one or more coupling structures (e.g., coupling structure 438) extending from an interior surface of the major needle hub 306. The coupling structures may be configured to hold a portion of the first needle 310. The coupling structures may advantageously support the first needle 310 and allow the first needle 310 access to medication flowing through the dual needle apparatus 300 (e.g., through syringe assembly 502). The components of major needle attachment hub 306 will be described further with respect to FIG. 4.

Turning to minor needle attachment hub 308, minor needle attachment hub 308 may include a first portion 318 and a second portion 320. An end of first portion 318 proximate to major needle attachment hub 306 may have a diameter substantially similar to an end of the second portion 316 of the major needle attachment hub 306. From this end of first portion 318 proximate to major needle attachment hub 306, first portion 318 may taper until it merges with second portion 320. The first portion 318 may merge with an exterior surface of second portion 320, as will be described in more detail herein.

Second portion 320 extends from a central region of the major needle attachment hub 306. The central region may extend along a diameter of a transverse plane of first portion 318. The width of the second portion 320 may be substantially similar to the width of the central region. A length of the second portion 320 parallel to the transverse plane of the first portion 318 may be substantially similar to a diameter of the corresponding portion. The second portion 320 may include a central rectangular portion and two curved ends.

While the above example describes the second portion 320 as including a central rectangular region with two curved edges, other forms are possible. As another example, the second portion 320 may be substantially cylindrical. For example, a diameter corresponding to an end of the second portion 320 may be substantially similar to a diameter of an end of the major needle attachment hub 306, such as an end corresponding to second portion 316.

The interior of the minor needle attachment hub 308 may include one or more coupling structures, such as coupling structure 438 of major needle attachment hub 306. The coupling structures may be configured to hold a portion of the second needle 312. Second needle 312 may, for example, be coupled at a horizontal distance from first needle 310 perpendicular to the longitudinal axis of the dual needle apparatus 300. The coupling structures may advantageously support the second needle 312 and allow the second needle 312 to release air and/or medication to an external environment (e.g., a hospital room, a doctor's office, a patient's home, etc.). The release of air or medication may release pressure caused by a flow of medication through the first needle 310, as will be described with respect to FIGS. 5A-7. The components of minor needle attachment hub 308 will be described further with respect to FIG. 4.

The first needle 310 may include a needle shaft and bevel. A length of the first needle 310 may be greater than a length of the second needle 312. An end of the first needle interior to the major needle attachment hub 306 may connect with a cavity configured to allow the flow of medication from a syringe assembly 502 through the first needle 310, as will be described further with respect to FIGS. 5A-7. The first needle 310 may include, but is not limited to, stainless steel.

The second needle 312 may also include a needle shaft and a bevel. A normal vector extending from a planar surface of the bevel of second needle 312 may extend in a different direction than a normal vector extending from a planar surface of the bevel of first needle 312. An end of the second needle 312 may, in some examples, be coupled within the minor needle attachment hub 308 with one or more coupling structures, as will be described with respect to FIG. 4. The one or more coupling structures may allow second needle 312 access to an external environment through a vent (e.g., a hospital room, a doctor's office, a patient's home, etc.). The second needle 312 may facilitate the flow of air or medication through the vent, such as to release pressure caused by the flow of medication through the first needle 310, as will be described with respect to FIGS. 5A-7. The second needle 312 may include, but is not limited to, stainless steel.

FIG. 4 illustrates a view of an example dual needle apparatus 300 including an interior of the dual needle apparatus 300 in accordance with some aspects of the disclosure. FIG. 4 illustrates example interiors of body component 304, major needle attachment hub 306, and minor needle attachment hub 308. The dual needle apparatus 300 may include more (or fewer) elements than those shown in FIG. 4. It is not necessary, however, that all of these elements be shown in order to prescribe enabling disclosure.

As illustrated in FIG. 4, body component 304 includes flanges 303, interior cavity 404, and coupler 406. Flanges 303 may extend from an exterior of body component 304. As described with respect to FIG. 3, some flanges 303 may extend from both the exterior of body component 304 and major needle attachment hub 306. Flanges 303 may advantageously support dual needle apparatus 300 as medication flows through dual needle apparatus 300, as described herein. Flanges 303 may, for example, provide a resistive force to maintain the shape of dual needle apparatus 300 as medication flows through dual needle apparatus 300 as described herein. Interior cavity 404 may be a space within an interior surface of body component 304. The interior surface of body component 304 may form a smooth surface parallel to an exterior surface of body component 304. Coupler 406 may be inside interior cavity 404. For example, coupler 406 may fixedly coupled to the interior surface of body component 304.

Coupler 406 includes needle channel 408 and support structures 410. Needle channel 408 may allow first needle 310 to access medication flowing through the dual needle apparatus 300, such as from a syringe assembly 502, as described herein. Needle channel 408 may, in some examples, be coupled to a portion of first needle 310. For example, a portion of the first needle 310 may fit in a cavity provided by needle channel 408. The walls of needle channel 408 may provide a pressure against the needle to limit movement of the first needle 310. The pressure may facilitate the flow of medication through first needle 310 as described herein at least by providing a resistive force responsive to a flow of medication to facilitate first needle 310 remaining coupled to dual needle apparatus 300 as the medication flows through first needle 310. Needle channel 408 may, in some examples, also be coupled to coupling structure 438 as will be described herein with respect to components of the major needle attachment hub 306. Coupling structure 438 may, in some examples, extend into needle channel 408 such that needle channel 408 applies a resistive pressure to the coupling structure 438. The resistive pressure may provide a resistive force responsive to a flow of medication to facilitate coupling structure 438 remaining coupled to dual needle apparatus 300 as the medication flows through first needle 310.

One or more support structures 410 may surround needle channel 408. Illustratively, the one or more support structures 410 may be flanges extending from an exterior surface of needle channel 408. Alternatively, the one or more support structures 410 may form a substantially conical enclosure around needle channel 408. The one or more support structures 410 include a top portion 412, a central portion 414, and a bottom portion 416. Top portion 412 may, in some examples, couple to needle channel 408 (e.g., through adhesives, through applied pressure, etc.). Top portion 412 may additionally, or alternatively, couple directly or indirectly (e.g., through other structures) to an interior surface of body component 304. Central portion 414 may extend from top portion 412 to a bottom portion 416. Bottom portion 416 may be coupled to a bottom portion of needle channel 408 (e.g., through adhesives, through applied pressure, etc.).

Major needle attachment hub 306 includes first portion 314 and second portion 316, as described with respect to FIG. 3. Coupling structure 438 extends through first portion 314 into second portion 316. Coupling structure 438 may form an enclosure surrounding at least a portion of first needle 310. Coupling structure 438 may, for example, be coupled to at least the portion of first needle 310 with one or more adhesive materials. Coupling structure 438 may additionally, or alternatively, apply a mechanical pressure against the first needle 310 to couple with the first needle 310. The adhesion and/or pressure provided by coupling structure 438 may advantageously hold first needle 310 in place as medication flows through dual needle apparatus 300 through first needle 310, as described herein.

Major needle attachment hub 306 further includes vent component 418. Vent component 418 includes a vent 420 and a mounting structure 422. Mounting structure 422 may be coupled to an interior surface of major needle attachment hub 306, such as by adhesives, plastic welds, or the like. Mounting structures 422 may further be coupled to the exterior surface of vent 420 parallel to a longitudinal axis of the vent 420. Vent 420 may have one end exposed to an external environment (e.g., a hospital room, a doctor's office, a patient's home, etc.). The other end of vent 420 may be proximate to second needle 312. The other end of vent 420 may, for example, be coupled to an end of the second needle 312 so that a lumen of second needle 312 has contact with the interior cavity formed by vent 420. The second needle 312 can thereby allow a flow of air or medication to release to the external environment. The release of air or medication may correspond to a flow of medication through the first needle 310 such as to a medication bladder 1128, from a medication vial, or the like.

Minor needle attachment hub 308 may couple to major needle attachment hub 306, such as through one or more adhesives, plastic welding, another coupling method, or some combination thereof. As described with respect to FIG. 3, a first portion 318 of the minor needle attachment hub 308 may have a diameter substantially similar to an end of the second portion 316 of the major needle attachment hub 306. From this end of first portion 318 proximate to major needle attachment hub 306, first portion 318 may taper until it merges with second portion 320 of the minor needle attachment hub 308. The second portion 320 includes a central portion 432 and edge portions 434. Central portion 432 may surround first needle 310 and second needle 310. Central portion 432 may, for example, be coupled to first needle 310 and second needle 310 with one or more adhesives, pressure, other coupling techniques, and the like, or some combination thereof. Central portion 432 may thereby advantageously hold first needle 310 in place as medication flows through dual needle apparatus 300 through first needle 310, as described herein. Central portion 432 may further hold second needle 312 in place, thereby facilitating the release of air and/or medication from second needle 312 responsive to the flow of medication through first needle 310. Edge portions 434 may provide additional support to first needle 310 and second needle 312 during the flow of medication through first needle 310 by generating a pressure against central portion 432.

First needle 310 may include a bevel 440 and a shaft 442, the shaft 442 extending through major needle attachment hub 306 and minor needle attachment hub 308, as described herein. The shaft 442 of first needle 310 may, in some examples, extend, at least partly, into body component 304. Illustratively, the shaft 442 may extend into needle channel 408, as described herein. Bevel 440 may form an angle with respect to shaft 442. First needle 310 may include a hollow cavity (e.g., a lumen) to facilitate the flow of medication through dual needle apparatus 300 (e.g., from a syringe assembly 502, from a vial, etc.).

Second needle 312 may include a bevel 444 and may include a shaft 446. The shaft 446 may extend through minor needle attachment hub 308, as described herein. The shaft 446 of second needle 312 may, in some examples, extend, at least partly, into major needle attachment hub 306. Illustratively, the shaft 446 may extend into coupling structure 438, as described herein. Bevel 444 may form an angle with respect to shaft 446. Second needle 312 may include a hollow cavity (e.g., a lumen) to facilitate the flow of air and/or medication through the second needle 312 as medication flows through first needle 310, as described herein.

### Example Insertion of Dual Needle Apparatus Into a Medication Bladder

FIG. 5A illustrates a view 500 of an example dual needle apparatus 300 and the medication bladder 1128 in accordance with some aspects of the disclosure. As illustrated in FIG. 5A, dual needle apparatus 300 is coupled to syringe assembly 502. When coupled to syringe assembly 502, the dual needle apparatus 300 may be considered a component of syringe assembly 502. As described herein with respect to FIGS. 2-3, the syringe assembly 502 may further include a luer component configured to receive threads of luer component 302 of FIG. 3. A caregiver may, in some examples, align the threads of luer component 302 with corresponding internal threads of the luer component of the syringe assembly 502, such as by rotating at least one of dual needle apparatus 300 and syringe assembly 502 until a stopping point is reached. The stopping point may indicate that dual needle apparatus 300 and syringe assembly 502 have coupled to form a luer lock fitting, which may advantageously reduce or eliminate the likelihood of leaking of medication from a reservoir 506 of the syringe assembly 502 during a flow of medication from the reservoir 506 through the dual needle apparatus 300.

Reservoir 506 may be a substantially cylindrical enclosure with a diameter substantially similar to that of body component 304 of the dual needle apparatus 300. Reservoir 506 may alternatively be a substantially cylindrical enclosure with a diameter substantially similar to that of luer component 302 of the dual needle apparatus 300. Reservoir 506 may include a hollow cavity bounded by the cylindrical enclosure of reservoir 506. The hollow cavity may have a diameter than the exterior of the reservoir 506. For example, the hollow cavity may have a diameter less than two times the thickness of the cylindrical enclosure of reservoir 506. Reservoir 506 may be made of a material that is biocompatible and insulin compatible so that it does not degrade when exposed to insulin or other biological materials. Reservoir 506 may, in some examples, be made of a medical grade plastic (e.g., polypropylene (PP), etc.), a medical grade metal, and the like, or some combination thereof. As illustrated in FIG. 5A, the reservoir 506 includes a stopping portion extending radially outward from an edge of reservoir 506. The stopping portion may stop the plunger 508 from descending farther into reservoir 506, as described herein.

Plunger 508 may be aligned with a longitudinal axis of syringe assembly 502. As illustrated in FIG. 5A, the plunger 508 may form a substantially cylindrical structure with a diameter smaller than that of reservoir 506. The plunger 508 may include a gripping structure located at an end distal to the reservoir 506. The plunger 508 may further include a disk with substantially the same diameter as the hollow cavity of reservoir 506. The disk may serve to apply a pressure against air or medication included in reservoir 506. Illustratively, when the gripping portion of plunger 508 is pressed with a first force (e.g., by a caregiver, a patient, etc.). The plunger may then apply a force, corresponding the first force, against air or medication included in reservoir 506. In response, medication included in syringe assembly 502 may flow through the dual needle apparatus 300. The flow of medication through the dual needle apparatus 300 into medication bladder 1128 will be described in more detail with respect to FIG. 6.

FIG. 5B illustrates a view 520 of at least part of example dual needle apparatus 300 and a portion of the medication bladder 1128, in accordance with some aspects of the disclosure. View 520 may be a sectional view illustrating at least a portion of dual needle apparatus 300 passing into an interior 522 of inlet seal 202, where the interior 522 includes a main cavity 538. Interior 522 may be formed by the rigid portion 204 and flexible portion 208, as described with respect to FIG. 2.

The illustrated portion of medication bladder 1128 includes at least part of central region 211 including input port 200 and inlet seal 202. The illustrated portion of medication bladder 1128 further includes other structure 201 surrounding and/or supporting an input port 200. Supporting structures illustrated in FIG. 5B also include at least one structure 214, as described with respect to FIG. 2.

With respect to inlet seal 202, FIG. 5B illustrates that a supporting structure 532 may be configured to surround and support inlet seal 202. A top surface 536 of inlet seal 202 may have a diameter substantially similar to an exterior 540 of supporting structure 532. A body portion 539 of inlet seal 202 may have a diameter substantially similar to an interior 542 of supporting structure 532. The supporting structure 532 includes an angled edge, which forms an angled gap 534 with respect to inlet seal 202. The angled gap 534 may facilitate removal of inlet seal 202, such as for replacement, cleaning, or the like. However, in some examples, supporting structure 532 may not include an angled edge or angled gap 534.

The illustrated portion of dual needle apparatus 300 includes minor needle attachment hub 308, first needle 310, and second needle 312, as described with respect to FIGS. 3-FIG. 5A. The minor needle attachment hub 308 may be proximate to a top surface 536 of inlet seal 202. The minor needle attachment hub 308 may, for example, physically contact at least part of top surface 536. When the minor needle attachment hub 308 is in position (e.g., proximate to a top surface 536, in physical contact with at least a portion of top surface 536, etc.), at least a portion of the first needle 310 and at least a portion of second needle 312 may extend through interior 522, inlet seal 202, and interior 524 of medication bladder 1128. The first needle 310, as illustrated, extends into main cavity 538 of the medication bladder 1128 such that the bevel 440 is within main cavity 538. The second needle 312, as illustrated, extends at least partly into the interior 522 such that at least a portion of bevel 444 of the second needle 312 is within the interior 522.

Medication, in some examples, may flow through first needle 310 into main cavity 538. The flow of medication may generate a pressure against air and/or medication within main cavity 538. The pressure may cause the air and/or medication to leave medication bladder 1128 through second needle 312, as will be described further with respect to FIG. 6.

### Example Flows Relating to Movement of Medication With the Dual Needle Apparatus

FIG. 6 illustrates an example method 600 for delivery of medication to the interior of an example medication bladder. Some of the processes, steps, and/or modules discussed herein with respect to FIG. 6 may be combined, separated into sub-parts, omitted entirely, and/or rearranged to run in a different order and/or in parallel.

At block 602, a caregiver may access a medication bladder, such as medication bladder 1128 of FIGS. 2 and 5A-5B. As described with respect to FIGS. 2 and 5A-5B, medication bladder 1128 may include an enclosure having the capacity to store a quantity of medication. The enclosure of the medication bladder 1128 may, for example, have the capacity to store a quantity of insulin.

At block 604, the caregiver may insert a syringe assembly, such as syringe assembly 502 of FIG. 5A into an input port, such as input port 200 of FIG. 2 or FIG. 5B of medication bladder 1128. As discussed with respect to FIG. 5A, syringe assembly 502 may include a reservoir 506. The reservoir 506 may comprise the quantity of medication described at block 602. Illustratively, the reservoir 506 may include the quantity of insulin. Syringe assembly 502 may further include two needles of differential length, such as when coupled to dual needle apparatus 300. The two needles of differential length may, for example, be first needle 310 and second needle 312, where first needle 310 is longer than second needle 312 as described herein.

At block 606, the caregiver may cause at least the quantity of medication to move from the syringe assembly 502 into the medication bladder 1128. Illustratively, the caregiver may apply a force to plunger 508 of syringe assembly 502. The force may cause the plunger to apply a force to air and/or medication included in reservoir 506. This application of force may cause the medication to flow through dual needle apparatus 300, for example, through first needle 310. The application of force may, for example, cause insulin to flow through first needle 310 into the main cavity 538 of medication bladder 1128. The flow of insulin into medication bladder 1128 may apply a pressure against air or medication present in the main cavity 538. As this pressure builds, the pressure may cause air or medication to exit from the medication bladder 1128 through second needle 312. The air or medication may, for example, flow through second needle 312 through vent 420 into an external environment (e.g., a hospital room, a doctor's office, a patient's home, etc.)

By way of example, the medication bladder 1128 may initially be empty. The medication bladder 1128 may, for example, include air and not medication. The reservoir 506 of syringe assembly 502 may, in some examples, include a quantity of insulin. A caregiver may, in further examples, insert the syringe assembly 502 into the medication bladder 1128, as described with respect to FIGS. 5A-5B. The caregiver may apply a force to plunger 508 and cause insulin to flow from reservoir 506 through first needle 310 into main cavity 538. The flow of insulin may cause a corresponding pressure to generate inside of medication bladder 1128. The pressure may force at least some of the air towards second needle 312. Second needle 312 may be connected to an external environment through vent 420, as described with respect to FIG. 4, where the external environment may be of lower pressure than the medication bladder 1128. Accordingly, the pressurized air from the medication bladder 1128 may flow through the vent 420 into the external environment until an equilibrium is reached. This may occur continuously or substantially continuously as the caregiver continues to apply force to plunger 508. As the amount of air reduces in medication bladder 1128, in some examples, insulin may flow through the vent 420 into the external environment. In some examples, the insulin may even come into contact with a patient's skin. A caregiver may, in some examples, take the release of insulin as an indication that the medication bladder 1128 is substantially full, such that the quantity of air within the medication bladder is eliminated or within acceptable limits.

Method 600 is described herein as being implemented by a caregiver. However, method 600 may, in some examples, be implemented by a patient or the like. In some examples, method 600 may be implemented by some combination of a caregiver, a patient, and the like.

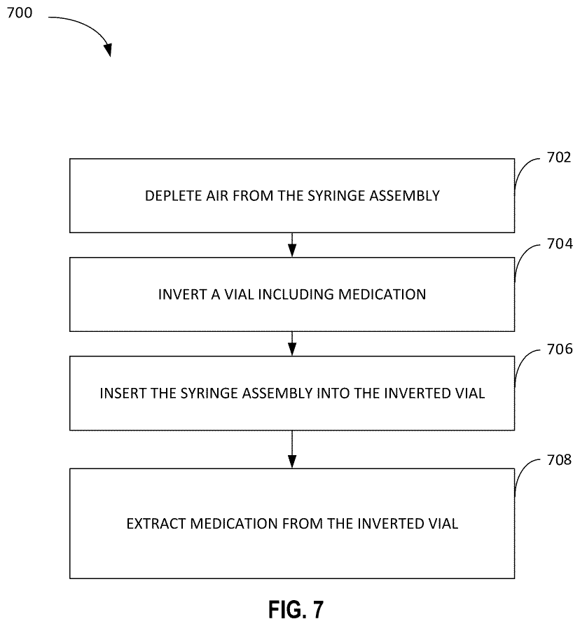

FIG. 7 illustrates an example method 700 for extraction of medication from a medication vial with an example dual needle apparatus 300. Some of the processes, steps, and/or modules discussed herein with respect to FIG. 4 may be combined, separated into sub-parts, omitted entirely, and/or rearranged to run in a different order and/or in parallel.

During execution of method 700, the dual needle apparatus 300 may, in some examples, be coupled to syringe assembly 502, as described with respect to FIG. 5A. At block 702, a caregiver may deplete air from the syringe assembly 502, such as by applying a force to plunger 508. The force may, for example, cause the plunger 508 to slide or otherwise move down into reservoir 506 until a stopping point is reached thereby expelling air from the reservoir 506.

At block 704, the caregiver may invert a vial including medication. The caregiver may illustratively invert a vial including insulin. The inversion of the vial may cause any air in the vial to flow to the top of the vial away from a needle insertion area of the medication vial. The needle insertion area may, in some examples, be made of a medical grade flexible material. In one example, the needle insertion area may be polyisoprene.

At block 706, the caregiver may insert syringe assembly 502 into the inverted vial. Illustratively, the caregiver may insert syringe assembly 502 into the inverted vial until minor needle attachment hub 308 is proximate to the needle insertion area of the medication vial. The minor needle attachment hub 308 may, in some examples, physically contact the needle insertion area of the medication vial. Once the minor needle attachment hub 308 is proximate to the needle insertion area of the medication vial, both first needle 310 and second needle 312 may be within the medication vial. The first needle 310 may extend deeper into the medication vial than the second needle 312. Both first needle 310 and second needle 312 may, for example, be in contact with medication inside the medication vial. However, in some examples, only first needle 310 may be within the medication vial. In further examples, first needle 310 may be in contact with medication inside the medication vial. As discussed with respect to block 704, the medication may be insulin.

At block 708, the caregiver may extract medication from the inverted vial. The caregiver may, for example, apply a force to plunger 508 that causes the plunger 508 to slide or otherwise move out of reservoir 506. A corresponding force, such as a vacuum force, may be applied against the medication in the vial. The caregiver may continue applying force until a desired amount of medication is present in the reservoir 506. The caregiver may, for example, continue applying force until a desired quantity of insulin is present in the reservoir 506. During the filling process air may enter the medication vial between at least one of the first needle 310 and the needle insertion area of the medication vial to balance the amount of medication exiting the medication vial into the syringe assembly 502, such as into the reservoir 506. Additionally, or alternatively, air may enter the medication vial through second needle 312 to balance the amount of medication exiting the medication vial into the syringe assembly 502.

Method 700 is described herein as being implemented by a caregiver. However, method 700 may, in some examples, be implemented by a patient or the like. In some examples, method 700 may be implemented by some combination of a caregiver, a patient, and the like.

### Terminology and Other Considerations

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. The terms “comprising,” “including,” “having,” and the like are synonymous and are used inclusively, in an open-ended fashion, and do not exclude additional elements, features, acts, operations, and so forth. That is, the above terms are to be interpreted synonymously with the phrases “having at least” or “including at least.” For example, when used in the context of a process, the term “comprising” means that the process includes at least the recited steps but may include additional steps. When used in the context of a device, the term “comprising” means that the device includes at least the recited features or components but may also include additional features or components. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conditional language, such as “can,” “could,” “might,” or “may,” unless specifically stated otherwise, or otherwise understood within the context as used, is generally intended to convey that certain examples include, while other examples do not include, certain features, elements, or steps. Thus, such conditional language is not generally intended to imply that features, elements, or steps are in any way required for one or more examples or that one or more examples necessarily include logic for deciding, with or without user input or prompting, whether these features, elements, or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount.

The term “and/or” as used herein has its broadest least limiting meaning, which is the disclosure includes A alone, B alone, both A and B together, or A or B alternatively, but does not require both A and B or require one of A or one of B. As used herein, the phrase “at least one of” A, B, “and” C should be construed to mean a logical A or B or C, using a non-exclusive logical or.

Any methods disclosed herein need not be performed in the order recited. The methods disclosed herein include certain actions taken by a practitioner; however, they can also include any third-party instruction of those actions, either expressly or by implication.

Conditional language used herein, such as, among others, “can,” “could,” “might,” “may,” “e.g.,” and the like, unless specifically stated otherwise, or otherwise understood within the context as used, is generally intended to convey that certain, certain features, elements and/or steps are optional. Thus, such conditional language is not generally intended to imply that features, elements, and/or steps are in any way required or that one or more implementations necessarily include logic for deciding, with or without other input or prompting, whether these features, elements and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain implementations require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

Any methods disclosed herein need not be performed in the order recited. The methods disclosed herein include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (for example, physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (for example, solid state storage devices, disk drives, etc.). The various functions disclosed herein may be embodied in such program instructions, and/or may be implemented in application-specific circuitry (for example, ASICs or FPGAs) of the computer system. Where the computer system includes multiple computing devices, these devices may, but need not, be co-located. The results of the disclosed methods and tasks may be persistently stored by transforming physical storage devices, such as solid-state memory chips and/or magnetic disks, into a different state. The computer system may be a cloud-based computing system whose processing resources are shared by multiple distinct business entities or other users.

In some embodiments, the filling and venting process may be monitored or controlled by a computer system, such as a smart medication pump, mobile application, or cloud-based platform. The system may record filling events, track medication usage, and provide reminders or alerts to the user. Sensors may be integrated into the syringe assembly or medication bladder to detect the presence of air, the completion of venting, or the volume of medication delivered. Automated control of the plunger or venting process may further enhance safety and accuracy, and may allow remote monitoring by healthcare providers. While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain implementations disclosed herein is indicated by the appended claims rather than by the foregoing description. All changes which come within the meaning and range of equivalency of the claims are to be embraced within their scope.

1.-14. (canceled)

15. A dual needle apparatus comprising:
a luer component configured to couple to a reservoir to form a luer lock fitting, wherein the reservoir comprises a quantity of medication; and
at least two needles of differential length,
wherein the at least two needles are coupled to the luer component,
wherein the at least two needles comprise a first needle longer than a second needle with respect to a longitudinal axis of the dual needle apparatus,
wherein, when the luer component is coupled to the reservoir, the first needle is able to access the quantity of medication,
wherein the first needle is configured to deliver at least a portion of the quantity of medication to a medication bladder,
wherein an end of the second needle is positioned proximate to a vent to an external environment such that the second needle is in fluid communication with an interior cavity of the medication bladder and with the external environment via the vent, and
wherein, when the first needle is delivering medication to the medication bladder, the second needle vents at least one of air or medication from the medication bladder to the external environment via the vent.

16. The dual needle apparatus of claim 15, wherein the medication comprises insulin.

17. The dual needle apparatus of claim 16, wherein the external environment comprises air of a room environment.

18. The dual needle apparatus of claim 17, wherein the first needle and the second needle are separated by a horizontal distance perpendicular to the longitudinal axis of the dual needle apparatus.

19. The dual needle apparatus of claim 18, wherein a vector extending from a bevel of the first needle extends in a different direction than a vector extending from a bevel of the second needle.

20. The dual needle apparatus of claim 19, wherein the luer component comprises a slip-tip fitting.

21. The dual needle apparatus of claim 20, wherein the apparatus is configured to couple to a syringe assembly having a reservoir volume between 1 mL and 10 mL.

22. The dual needle apparatus of claim 21, wherein the second needle is positioned within 5 mm of the luer component.

23. The dual needle apparatus of claim 22, wherein the apparatus is disposable.

24. The dual needle apparatus of claim 23, wherein the vent comprises a channel extending from the second needle to an exterior surface of the apparatus.

25. The dual needle apparatus of claim 24, wherein the apparatus is operable to deliver at least one of insulin, glucagon, or another medication.

26.-31. (canceled)

32. A method comprising:
inserting a syringe assembly into an input port of a medication bladder, the syringe assembly comprising:
a dual needle apparatus comprising a first needle and a second needle, the first needle longer than the second needle; and
a reservoir comprising a quantity of medication;
causing at least the quantity of medication to flow from the reservoir to the medication bladder through the first needle,
wherein the second needle facilitates a release of pressure caused by the flow of medication through the first needle into the medication bladder at least by facilitating the release of at least one of air or medication to an external environment via a vent in fluid communication with the second needle.

33. The method of claim 32, wherein the medication comprises insulin.

34. The method of claim 33, wherein inserting the syringe assembly into the input port of the medication bladder comprises inserting the dual needle apparatus through an inlet seal coupled to the input port,
wherein after insertion the first needle is in communication with an interior cavity of the medication bladder and the reservoir, and
wherein the second needle is in communication with the interior cavity of the medication bladder and with the external environment.

35. The method of claim 34, wherein insulin is released to an external environment through the second needle.

36. The method of claim 35, wherein the released insulin contacts an exposed area of skin of a patient.

37. The method of claim 36, wherein the quantity of medication is greater than a storage capacity of the medication bladder.

38. The method of claim 37, wherein the external environment comprises air of a room environment.

39. The method of claim 38, wherein the dual needle apparatus is disposable.

40. A method comprising:
depleting air from a syringe assembly, the syringe assembly comprising:
a dual needle apparatus comprising a first needle and a second needle, the first needle longer than the second needle; and
a reservoir configured to hold a quantity of medication;
inserting the syringe assembly into a vial, the vial comprising at least the quantity of medication; and
extracting the medication from the vial to the reservoir through the first needle,
wherein, when extracting the medication from the vial, the second needle is configured to facilitate movement of the medication from the vial at least by allowing air from an external environment to pass into the vial during extraction, the air creating a pressure against the medication, wherein the air passes into the vial via a vent in fluid communication with the second needle.

41.-45. (canceled)
